(19) United States
(12) Patent Application Publication
(10) Pub. No.: US 2009/0163825 A1
(43) Pub. Date: Jun. 25, 2009

(54) Non-Invasive Method and System of Signaling a Hyper or Hypoglycemic State

(75) Inventors: Robert Hirsh, Merion Station, PA (US); Marc C. Torjman, Southampton, PA (US)

Correspondence Address: COZEN O'CONNOR, P.C., 1900 MARKET STREET, PHILADELPHIA, PA, 19103-3508, US

(73) Assignee: THE COOPER HEALTH SYSTEM, Camden, NJ (US)

(21) Appl. No.: 11/960,186

(22) Filed: Dec. 19, 2007

(51) Int. Cl. A61B 5/00

(52) U.S. Cl. 600/532

(57) ABSTRACT

A method and system for determining a hyper or hypoglycemic state in a diabetic patient using acetone concentration in exhaled breath by means of a nasal device containing an acetone sensor. When a hyper or hypoglycemic state is detected, an alarm is issued. The alarm can be audible and/or can be sent wirelessly to a local or remote computer system.

### BACKGROUND OF THE INVENTION

This invention relates to medical management of patients with diabetes. More particularly this invention relates to the field of hypoglycemia and hyperglycemia alarm systems.

Individuals with diabetes carry the risk of low blood sugar, known as hypoglycemia, usually resulting from an imbalance between food, exercise, and medications, wherein a low blood sugar reaction can cause disorientation, unconsciousness, and sometimes death. If not properly monitored, an individual or medical professional may not be aware that the individual has reached a hyper or hypoglycemic state.

Individuals with diabetes may also be at risk for hyperglycemia, which is an excess of sugar in the blood, and if the individual is not aware of the hyperglycemic state, serious damage and possibly death may ensue.

Diabetic ketoacidosis (DKA) is an acute, major, life-threatening complication of diabetes which mainly occurs in individuals with type 1 diabetes, and less often with type 2 diabetes. DKA is an acute state of severe uncontrolled diabetes that requires emergency treatment with insulin and intravenous fluids. DKA involves an increase in the serum concentration of ketones greater than 5 mEq/L, a blood glucose level of greater than 250 mg/dL although it is usually much higher), blood pH of less than 7.2, and a bicarbonate level of 18 mEq/L or less.

Hypo and hyperglycemia occur in both adults and children, and the risk depends on the degree and progression of the diabetic state. The risk is especially acute during the night or at other times when the individual is sleeping. Because of the life threatening effects of nocturnal hypoglycemia in young children, parents often experience nights with severe anxiety resulting in sleep deprivation for both parents and children trying to monitor the condition.”

Several automated, programmable, continuous glucose monitoring systems that use subcutaneous sensors have become available for diabetic ambulatory patients. For example, diabetic patients can now wear automated, programmable, continuous monitoring systems such as the two FDA approved glucose monitoring systems by Medtronic (Guardian REAL-Time System), and DexCom (STS Continuous Glucose Monitoring System). Those two systems are equipped with out of range patient alarms to notify the patient of large or dangerous deviations in blood glucose. The detection of hypoglycemia allows the patient to take corrective action to prevent potentially devastating complications. Choleau et al. have described the use of a continuous amperometric glucose sensor implanted in rats to predict hypoglycemia (Prevention of Hypoglycemia Using Risk Assessment With a Continuous Glucose Monitoring System, Diabetes 51:3263-3273, 2002).”

The relationship between acetone in exhaled breath and diabetes has been known for some time. For example, Melker, U.S. Pat. No. 6,981,947, assigned to U. of Fla. Research Foundation, Inc., disclosed in Example III measuring endogenous and exogenous compounds such as acetones in exhaled breath, stating that “normally, the exhaled breath of a person contains water vapor, carbon dioxide, oxygen, and nitrogen, and trace concentrations of carbon monoxide, hydrogen and argon, all of which are odorless.” Melker disclosed a sensor to be used as a sensitive detector for these odorants and for the diagnosis of tooth decay, gum disease or a variety of oral, pulmonary and sinus conditions.

Among the vapor phase odorant compounds detected by Mekler are acetone, which is present in diabetics who are in ketoacidosis, and the use of exhaled breath sensing as a highly sensitive method of diagnosing and following the course of treatment of this disease.

However, Mekler did not teach or suggest a system for continuously or periodically measuring acetone and using the measured data to calculate and alarm a hyper or hypoglycemic state.

Fu, U.S. Pat. No. 7,076,371, disclosed a non-invasive diagnostic and monitoring method and system based on odor detection. Fu's system is designed to emulate a biological nose, with numerous sensors, each with a different type of polymer responding differently to various odorant molecules. Fu taught an aerogel with a very large surface area for coating of a polymer, effectively simulating the huge number of same-type biological olfactory cells and their combined response. The polymer provides the necessary electronic and chemical coupling and a piezoelectric crystal is used for the quantitative conversion of trace amounts of odorant molecules to frequency-shift signals and allows detection of the markers of diabetes, for example. Fu also disclosed a heater incorporated with the detector to “refresh” the system so to provide confirmation of the detection of a targeted substance, which Fu said is important for minimizing false positive alarms, thus improving the general reliability of the system. Fu disclosed alternative types of sensors such as GC, HPLC, mass spectrometry, and conducting nanotubes whose conductivity changes as a result of gas absorption. Fu disclosed the relationship between acetone on a person's breath and ketoacidosis.

Other systems intended primarily for use outside of a hospital setting are designed to measure temperature and moisture level of an individual's skin and to determine insulin reaction based on changes in such temperature and moisture. The “Sleep Sentry” is designed to detect hypoglycemia by monitoring temperature and moisture level of the skin of a sleeping diabetic person. If a temperature drop or increased perspiration is detected, an alarm sounds and the wearer of the device is supposed to check his or her blood sugar.

Allen, et al., disclosed in U.S. 2004-0236244 A1 a hand-held medical apparatus for measuring acetone in exhaled breath as an indicator of ketosis and an aid for detection of weight loss via fat metabolism.

Cranley, et al., disclosed in U.S. 2005-0084921 A1 an enzyme based sensor coupled to a detectable signal mediator for measuring acetone and methods for using the sensor to detect disease, weight loss, and bioavailability monitoring of therapeutics.

Although the relationship between acetone in the breath and hypoglycemia in a diabetic is known, and methods for measuring acetone in a person's breath are known, no one has previously suggested a hypoglycemia and hyperglycemia alarm system based on changes in acetone levels in breath.

### SUMMARY OF THE INVENTION

It is an object of the present invention to provide an improved warning system for sleeping diabetics, a system which is not based on body temperature or skin moisture changes.

It is another object of the present invention to provide an improvement to existing total glycemic control (TGC) procedures by continuously or periodically measuring exhaled breath of a diabetic patient and determining presence of acetone, which is indicative of a hyper or hypoglycemic state of the patient.

Another object of the invention is to detect a hyper or hypoglycemic state in an individual and to provide an alarm when such state is detected.

A further object of the invention is to provide a system which can be used when a diabetic individual is sleeping and at risk of hyper or hypoglycemia, which is minimally invasive and does not require blood analysis, but can detect hyper or hypoglycemia and signal an alarm when hyper or hypoglycemia is detected.

In one aspect, the invention comprises (A) continuously or periodically determining acetone concentration in exhaled breath of a diabetic patient, (B) determining presence of a hyper or hypoglycemic state of the patient by calculating changes in the acetone concentration, and (C) issuing an alarm when the hyper or hypoglycemic state is determined.

In another aspect, the invention comprises a system for signaling a hyper or hypoglycemic state comprising means to continually or periodically measure acetone concentration in exhaled breath comprising a sensor fluidly connected with a subject's nasal passage, for example an infrared sensor, and a programmed controller which can calculate changes in the acetone concentration in the exhaled breath based on data received from the sensor, the controller programmed to determine presence of a hyper or hypoglycemic state based on the calculated changes in acetone concentration, and an audible and/or visual alarm when the presence of a hyper or hypoglycemic state is determined.

The present invention provides continuous measurement of acetone, a volatile metabolite of fat breakdown, in end-tidal gas as a proxy for hyper or hypoglycemic measurement. The invention would not substitute for periodic blood glucose determinations, but would complement glucose testing in a continuous or semi-continuous non-invasive fashion. It would help avoid hyper or hypoglycemic episodes and neurologic damage.

A system which includes plastic tubing, referred to herein as a cannula, which includes one or two nasal extensions and preferably an oral extension adapted to be worn in a subject's nose and mouth, fluidly connected to a source of vacuum, which draws small amounts of exhaled breath from the subject's nose and/or mouth, can be used to obtain samples of the subject's breath. In some embodiments, a thermistor or other temperature measuring device can be used to detect a condition where the cannula extensions are no longer properly placed in the nose and/or mouth, for example if the subject pulls the cannula out while sleeping or the cannula falls out for some other reason. The temperature measuring device or thermistor is electrically connected to the controller and changes in temperature are detected and interpreted. If the temperature shows periodic physiologic variation with respect to time, then the system is properly functioning and the acetone data are valid.

In some embodiments acetone detection is carried out in or adjacent to the nostrils and/or mouth and no tube, cannula, or vacuum is used.

### BRIEF DESCRIPTION OF THE DRAWINGS

FIG. 1 is a side perspective view of a cannula embodiment having nasal and oral extensions, with separate tubes for receiving breath exhaled through the mouth and nose.

FIG. 2 is a perspective view of a cannula having a nasal module and an acetone detection module within a portion of a tube fluidly connected to the module.

FIG. 3 is a perspective view of an embodiment of a system of the invention illustrating a cannula on a person's head and a module providing a source of vacuum and acetone level analysis.

FIG. 4 is a perspective view of an embodiment of a system of the invention showing a cannula in place receiving breath exhaled from a person's nose, tubing, source of vacuum, an analytical module, and an alarm.

FIG. 5 is a perspective view of an embodiment of a module for detecting acetone levels in exhaled breath which does not include tubing.

FIG. 6 is a perspective view which illustrates an embodiment of a module for receiving and combining oral and nasal exhaled breath and for connection to a vacuum source.

FIG. 7 illustrates an embodiment of a module for receiving exhaled nasal and oral breath.

FIG. 8 is an embodiment of a cannula which includes nasal and oral extensions, a thermistor on the oral extension, and a connector connected to a base station which includes a source of vacuum, an infrared gas analyzer, a programmed computer which analyzes data received from the gas analyzer and the thermistor, and an alarm which can issue an audible and/or visual alarm signal.

FIG. 9 is a process flow chart illustrating an embodiment of the method of the invention.

### DETAILED DESCRIPTION

While the invention is capable of being carried out in various embodiments, a few illustrative embodiments will be described in the following detailed description with reference to the drawings.

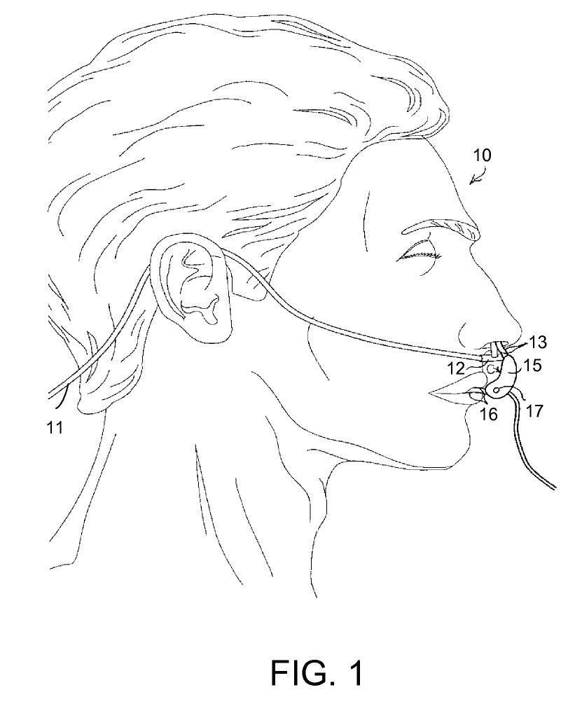

Referring first to FIG. 1, an embodiment of a system of the invention comprising a tube 11, a module 12 having nasal projections 13, a second module 15 fluidly attached to a mouth projection 16 and a thermistor 17 is illustrated on a person 10.

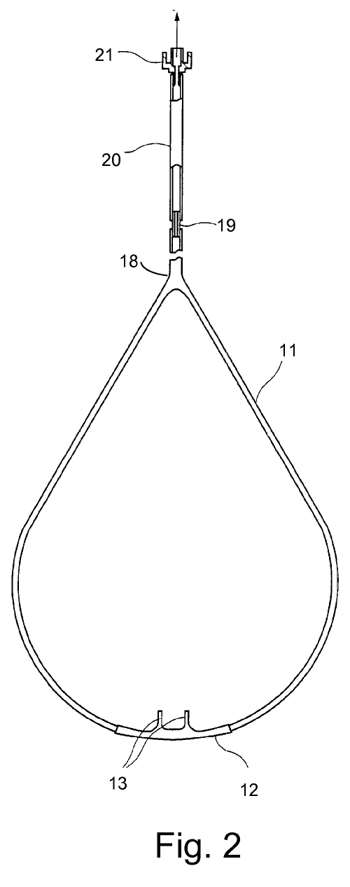

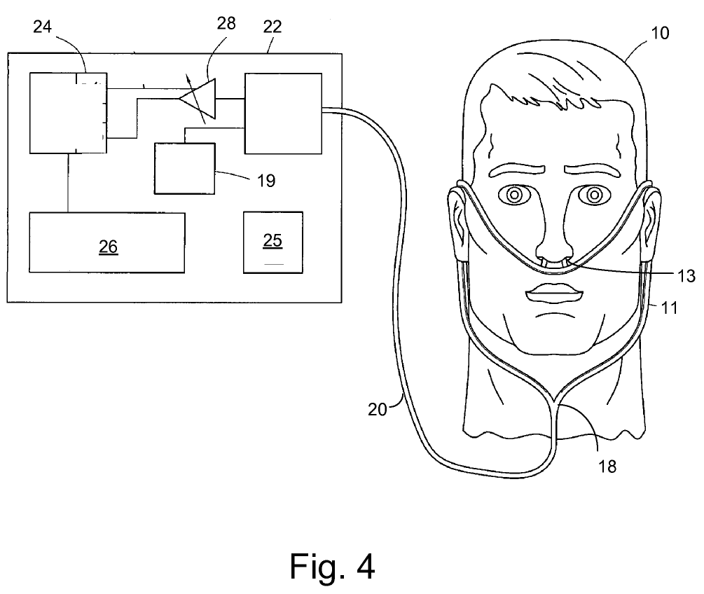

Referring now to FIG. 2, an embodiment with nasal projections 13 on a module 12 connected at each side to tube 11 joined at juncture 18 and including an acetone detection module 19 within a tube section 20 leading to a vacuum source 21 is illustrated in perspective. The system of FIG. 2 is adapted to receive breath from a sleeping person, usually a diabetic, exhaled from nostrils and drawn through tube 11 and then tube section 20 by a slight negative pressure resulting from vacuum source 21 and over the acetone detection module 19 which is within tube section 20 in this embodiment. The acetone detection module is arranged to allow expired breath to flow over and around the acetone detector 19. The acetone detector 19 comprises a modified infra red detector which is electronically connected to a controller 24 (FIG. 4), which calculates acetone level changes over time and is programmed to signal an alarm indicative of a dysglycemic condition, which activates an alarm 25 (FIG. 4) if the preselected threshold is exceeded. The thresholds are determined experimentally for the particular embodiment of the invention, and are adapted to signal either a hypoglycemic or hyperglycemic state. The acetone level considered normal and not exceeding a threshold may vary among individuals and so in some embodiments the threshold(s) can be set after determining non-hypoglycemic and non-hyperglycemic acetone levels for the individual.

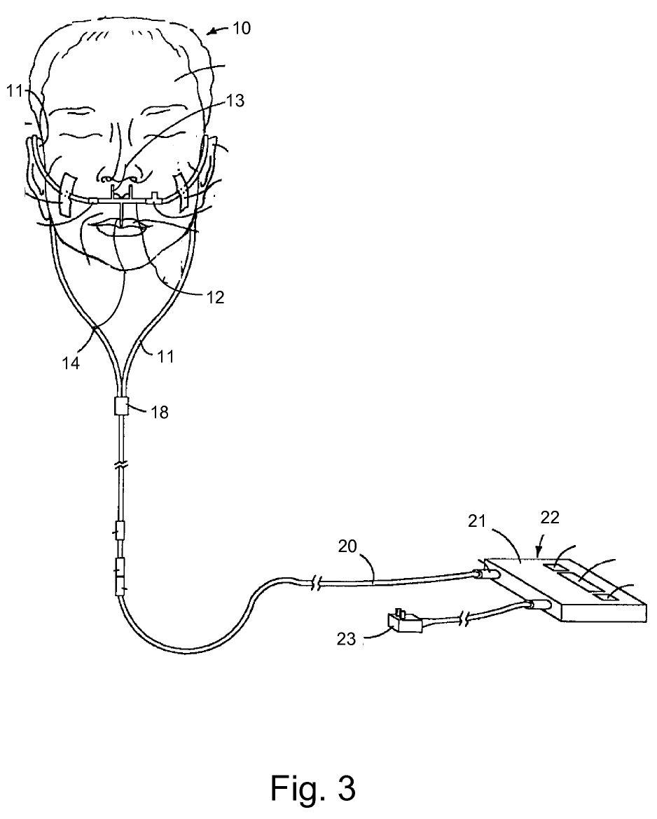

FIG. 3 is a perspective view of a sleeping individual 10 using a device of the invention having a tube 11 joined to a module 12 having nasal projections 13 and a mouth projection 14 for receiving breath exhaled through the nose and mouth and drawn through tube 11, juncture 18, tube section 20 and into module 22 which contains an acetone level analyzer, programmed controller, and power supply. The power supply can be either a battery or can be a transformer, in which case the device is plugged into a house power receptacle with plug 23. In some embodiments both house power and battery backup are employed in order to continue protection if the plug is accidentally withdrawn or in the event of a power failure.

FIG. 4 illustrates the person 10 with nasal projections extending directly from tube 11 rather than from a module 12 (FIG. 1). Again the tube 11 is joined at a juncture 18 which forms a single tube section 20 leading to a module 22 having a vacuum source 21, an acetone level detection module 19, a controller 24, a power supply 26, and an alarm 28 which sounds when the controller calculates and thereby determines that the acetone level has exceeded a dysglycemia threshold. Various alarm embodiments are contemplated, for example a strobe light in addition to sound can be provided for use in the case of a deaf individual, or a system to make a telephone call or internet message can be provided so that an off site person can be informed when a threshold has been exceeded.

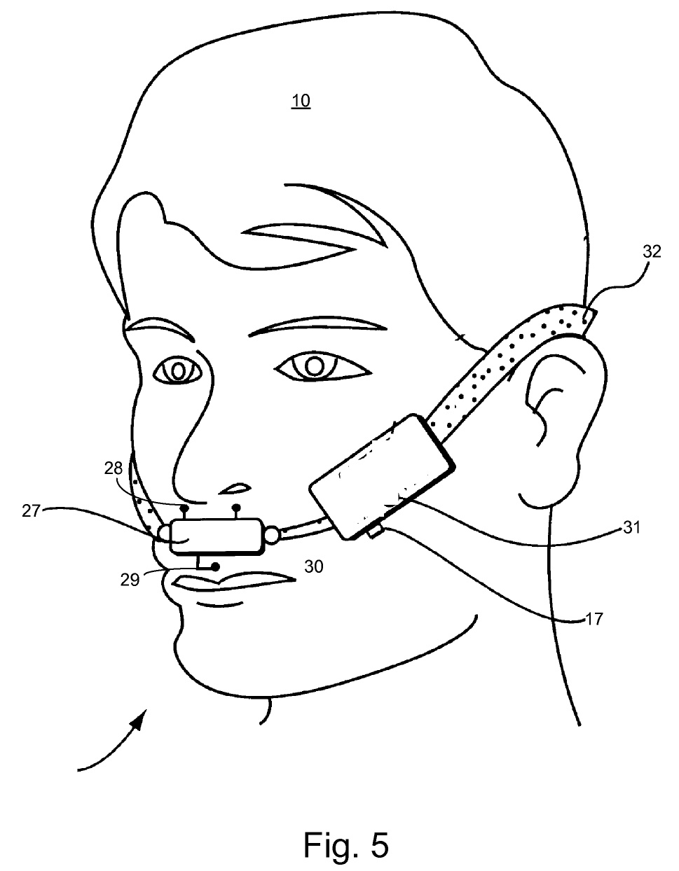

FIG. 5 illustrates another embodiment of the invention which does not include tubes 11, 20 (FIG. 1) or a vacuum source 21 (FIG. 1) but rather includes nasal detectors 28 below the person's 10 nostrils and an oral detector 29 above the person's mouth, the detectors 28, 29 having built in acetone analytical receptors mounted on tubeless module 27 which includes a battery power supply, a controller, and electrical connection 30 to an alarm module 31 mounted on a strap 32 which extends around the person's 10 head. In this embodiment no vacuum or tubing is necessary since the acetone level is measured and determined with the receptors 28, 29 and module 27. There are several chemical analyzers which are available in miniature sizes which can be used in embodiments such as the one illustrated in FIG. 5.

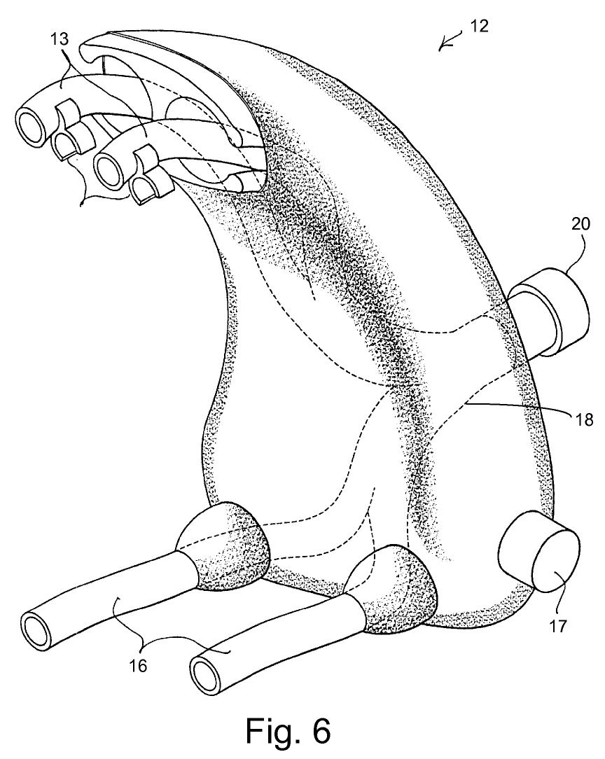

FIG. 6 illustrates in perspective view partially in phantom a module 12 having a nasal projection 13 and mouth projections 16, with a tube 20 leading to a vacuum source 21 (FIG. 2). The mouth and nasal breath receptors are joined at junction 18 within module 12. In this embodiment a thermistor 17 is used to determine temperature of breath flowing through the tubes, for example at juncture 18, and the associated controller 24 (FIG. 4) is programmed to send an alarm if the temperature of the breath in the tube system falls below a predetermined threshold which is indicative of the module not receiving actual breath but receiving room temperature air instead. This embodiment is designed to assure that the module does not become dislodged and that the person's exhaled breath is being received properly.

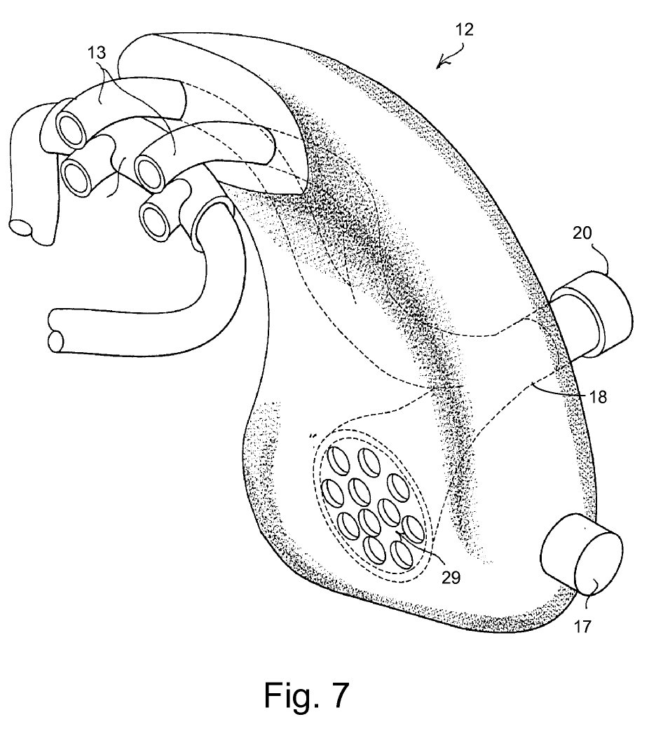

FIG. 7 is a different embodiment of module 12 wherein the oral detector 29 is a group of holes designed to be located just outside the person's mouth rather than an extension which goes into the person's mouth.

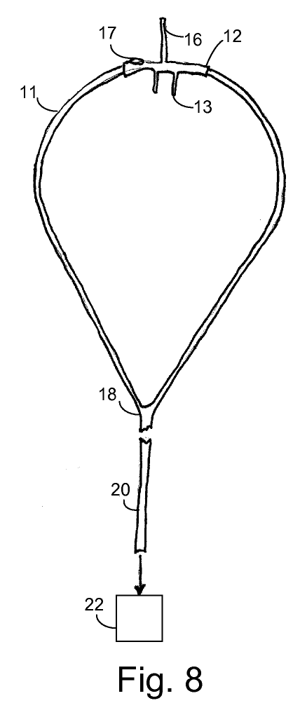

FIG. 8 illustrates an embodiment of the invention having module 12 which has a single mouth projection 16 and two nasal projections 13 for receiving exhaled breath, connected at each side of the module 12 to tube 11 which is then joined at juncture 18 and connected via tube section 20 to module 22 which contains a vacuum source, a controller, an acetone level analyzer, and power supply.

The acetone analyzer 19 can be a standard infrared gas analyzer 19 set up to detect small quantities of acetone in the exhaled breath of a diabetic individual.

The devices of the invention can be made in a range of sizes to fit an adult down to an infant. The device can be made of suitable plastics materials, known to those skilled in the art. The body and tubes may be made of similar or different plastics materials.

When the patient is ready to sleep, a start button is pressed. The next morning the patient presses the stop button to end the breath monitoring.

In the following discussion and in the claims, the terms “including” and “comprising” are used in an open-ended fashion, and thus should be interpreted to mean “including, but not limited to . . . ”. Also, the term “connect” or “connects” is intended to mean either an indirect or direct connection. Thus, if a first device connects to a second device, that connection may be through a direct connection, or through an indirect connection via other devices. The term “cannula” refers to a respiratory mask (either full or partial) that fluidly couples one or more of a patient's airways to a testing device. Thus, a “nasal cannula” couples at least one naris to the test device. Likewise, an “oral cannula” may couple to a patient's mouth. The word “cannula” alone could thus refer to a nasal cannula, an oral cannula, or a cannula that couples to both a patients nose and mouth.

The controller 24 may comprise a processor which may be a microcontroller, and can have an on-board converter A/D, D/A converter, on-board random access memory (RAM), read only memory (ROM), as well as other on-board circuits, such as circuits that allow the processor to communicate to external devices. The controller 24 may actually be more than one processor but must be programmed to carry out the acetone level detection and alarm functions. The controller 24 may also drive an indicator or display device coupled to the processor, and may be coupled to on and off switches.

As the person 10 inhales, at least a portion of the airflow into the nostrils is drawn through the tubing. The controller can be programmed to recognize the breathing cycles and to determine whether the airflow is due to exhaling or inhaling.

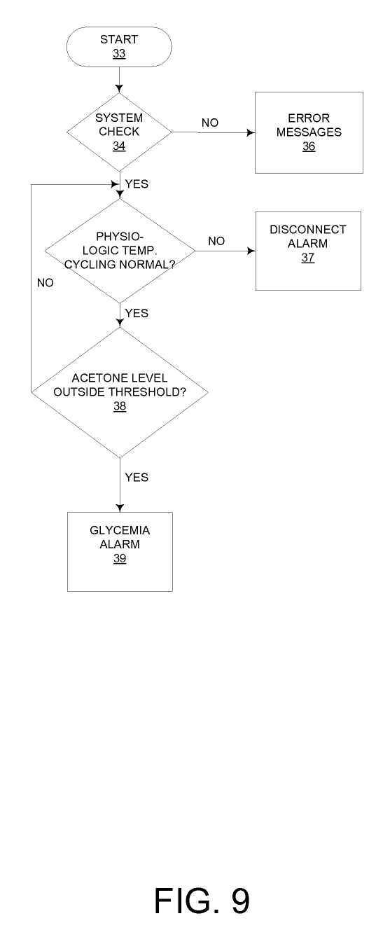

FIG. 9 illustrates a method in accordance with embodiments of the invention wherein the system is placed on the person so that nasal and/or oral breath is received and the device is switched on, the system starts 33 with an system check 34 to assure that temperature data is being received from the thermistor 17 (FIG. 1) and acetone levels are being received from acetone detector 19 (FIG. 2), otherwise an error message 36 is issued. If the system is working normally, if the temperature drops below a predetermined threshold, a temperature alarm 37 is issued to indicate that breath is not being received properly. If temperature is normal 37 and acetone levels are within a predetermined range, then the system signals normal operation. If the measured acetone level goes outside of a predetermined range 38, then dysglycemia alarm 39 is issued, indicating either hypoglycemia or hyperglycemia.

While the invention has been described and illustrated in detail herein, various alternatives and modifications should become apparent to those skilled in this art without departing from the spirit and scope of the invention.

What is claimed is:

1. A system for signaling a dysglycemic state comprising
means to continually or periodically measure acetone concentration in exhaled breath comprising sensor means and means to maintain the sensor means in a nasal passage,
means to process acetone concentration data and calculate changes in the acetone concentration in the exhaled breath,
means to determine presence of a dysglycemic state based on the calculated changes in acetone concentration, and
means to issue an alarm when the presence of a dysglycemic state is determined.

2. The system of claim 1 wherein the means to continually or periodically measure acetone concentration comprises a nasal projection, a cannula tube, a vacuum source, a controller, an acetone analyzer, and an alarm, the system adapted to establish a continual or periodic negative pressure at the nasal projection, to conduct exhaled breath to the acetone analyzer, and to continually or periodically analyze acetone concentration in the exhaled breath.

3. The system of claim 1 wherein the alarm means is an audible or visual signal.

4. The system of claim 1 wherein the means to continually or periodically measure acetone concentration comprises a mouth projection adapted to receive exhaled breath.

5. The system of claim 1 including means to measure temperature of the exhaled breath and means to compare the measured temperature to an expected temperature and thereby determine if the system is receiving exhaled breath.

6. The system of claim 1 including means to measure temperature of the exhaled breath, means to compare the measured temperature to an expected temperature and thereby determine if the system is receiving exhaled breath, and means to issue an audible or visual disconnect alarm if the measured temperature does not vary physiologically with respect to time.

7. The system of claim 1 including means to determine periodicity of acetone concentration with respect to a respiratory cycle.

8. The system of claim 1 including means to measure acetone concentration in end-tidal alveolar gas, means to calculate if the measured acetone concentration in the end-tidal alveolar gas exceeds a predetermined threshold, and means to signal a dysglycemic state if the calculated acetone concentration exceeds the predetermined threshold.

9. The system of claim [8] further including means to determine the degree to which the calculated acetone concentration exceeds the predetermined threshold and means to signal the degree of dysglycemic state based on the degree to which the acetone concentration exceeds the predetermined threshold.

10. The system of claim 1 wherein the means to measure acetone concentration comprises a thermistor adapted to measure temperature within the cannula, the system comprising means to compare the measured temperature to an expected temperature and thereby determine if the cannula is physiologically connected to the patient.

11. The system of claim 2 comprising means to determine changes in acetone concentration as a function of respiration and to compare changes in acetone concentration to expected changes in acetone concentration due to respiration.

12. The system of claim 2 further including means to measure CO₂ concentration in the exhaled breath, means to compare the measured concentration to expected concentration, and means to signal an error if the measured concentration varies from the expected concentration by a predetermined value.

13. A process comprising (A) continuously or periodically determining acetone concentration in exhaled breath of a diabetic patient, (B) determining presence of a dysglycemic state of the patient by calculating changes in the acetone concentration, and (C) issuing an alarm when the dysglycemic state is determined.

14. The process of claim [13] comprising determining changes in acetone concentration as a function of respiration and to compare changes in acetone concentration to expected changes in acetone concentration due to respiration.

15. The process of claim [13] comprising measuring CO₂ concentration in the exhaled breath, comparing the measured concentration to expected concentration, and signaling an error if the measured concentration varies from the expected concentration by a predetermined value.

16. The process of claim [13] comprising measuring temperature in exhaled breath and comparing measured temperature to an expected temperature and thereby determine if expired breath is being received.

17. The process of claim [13] comprising measuring temperature, comparing the measured temperature to an expected temperature, and thereby determining if exhaled breath is being received, and further comprising signaling an error or disconnect if the measured temperature does not vary physiologically with respect to time.
